(19) United States
(12) Patent Application Publication
(10) Pub. No.: US 2021/0386985 A1
(43) Pub. Date: Dec. 16, 2021

(54) BIODEGRADABLE MICRONEEDLES FOR TRANSDERMAL THERAPEUTIC AGENT DELIVERY

(71) Applicant: THE REGENTS OF THE UNIVERSITY OF CALIFORNIA, Oakland, CA (US)

(72) Inventors: Alireza Khademhosseini, Los Angeles, CA (US); Wujin Sun, Los Angeles, CA (US)

(73) Assignee: THE REGENTS OF THE UNIVERSITY OF CALIFORNIA, Oakland, CA (US)

(21) Appl. No.: 17/286,443

(22) PCT Filed: Oct. 28, 2019
(86) PCT No.: PCT/US2019/058333

§ 371 Date: Apr. 16, 2021

Related U.S. Application Data
(60) Provisional application No. 62/753,522, filed on Oct. 31, 2018.

(51) Int. Cl. A61M 37/00; A61K 9/00; A61K 9/70

(52) U.S. Cl. CPC A61M 37/0015; A61K 9/0021; A61K 9/7038; A61M 2202/0007; A61M 2037/0053; A61M 2037/0046; A61M 2202/0445; A61M 2037/0061

(57) ABSTRACT

A microneedle patch is described that can be used for the sustained delivery of therapeutic agents into living tissue (e.g., skin). The polymer (gelatin methacryloyl (GelMA)) patch can adjust delivery rates based on the degree of crosslinking. The anticancer drug Doxorubicin (DOX) was loaded into GelMA microneedles using a molding fabrication technique. The GelMA microneedles efficiently penetrated the stratum corneum layer of a mouse cadaver skin. Mechanical properties and therapeutic agent release behavior of the GelMA microneedles can be adjusted by tuning the degree of crosslinking. The efficacy of the DOX released from the GelMA microneedles was tested and demonstrated the anticancer efficacy of the released drugs against melanoma cell line A375. Because GelMA is versatile material in engineering tissue scaffolds, GelMA microneedles can be used as a platform for the delivery of various types of therapeutic agents to tissue.

### RELATED APPLICATION

This application claims priority to U.S. Provisional Patent Application No. 62/753,522 filed on Oct. 31, 2018, which is hereby incorporated by reference in its entirety. Priority is claimed pursuant to 35 U.S.C. § 119 and any other applicable statute.

### TECHNICAL FIELD

The technical field generally relates to biocompatible and biodegradable microneedles. More particularly, the technical field relates to a patch that incorporates microneedles for the sustained delivery of therapeutic agents into mammalian skin tissue and other uses.

### BACKGROUND

Skin is the largest organ of the human body and constitutes approximately 15% of total human body weight. It is composed of multiple layers including epidermis, dermis, and hypodermis. The outermost layer of the epidermis, stratum corneum, functions as the skin barrier, which also limits the availability of therapeutics for target areas beneath the epidermis. Compared with traditional hypodermic needle-based drug delivery, microneedles are small enough not to be visible to the naked eye and hence are friendly to patients with needle phobia. Compared with sonophoresis or iontophoresis-based transdermal drug delivery that needs dedicated electronic devices, microneedle patches are easy to apply without the prerequisite of requiring complex devices. Due to their microscale dimensions microneedles can puncture the skin seamlessly and can deliver a range of therapeutic molecules including ones with a wide range of molecular weights, such as small molecules, biomacromolecules, and even nanoparticles. Compared with chemical formulations for enhanced transdermal drug delivery, such as lipid nanoparticles, manufacturing of microneedles requires much lower cost and has a high batch to batch reproducibility. In addition, the depth of microneedle penetration could be arranged to solely penetrate the epidermis without damaging neurons in the dermis, minimizing the pain associated with transdermal drug delivery.

Microneedle-mediated transdermal drug delivery requires: (1) the material to have sufficient mechanical strength to penetrate the skin barrier; (2) the material to be biocompatible and not to cause irritation or other immune reactions after the application; (3) the material needs to dissolve or bio-degrade while releasing its payload; (4) the release profile of the drug should be slow and uniform to provide a sustained release of the drug for extended period of time. Generally, the polymer and casting medium characteristics including water solubility, molecular weight, viscosity, concentration, and entrapped air would affect the mechanical strength, skin insertional force, drug loading and the stability of microneedles. Different kinds of materials including polyvinyl alcohol, dextran, chitosan, alginate, carboxymethyl cellulose, maltose, dextrin, chondroitin sulphate, acrylate polymers, poly (β-ester), polylactide and sugars have been used to construct dissolvable microneedles. Most of these are water soluble and dissolve upon contact with the skin, hence are promising materials for encapsulation and delivery of drugs. However, the dissolution rate of most of these materials is quite fast resulting in an initial burst release that creates elevated levels of drug at often toxic levels. Controlling the dissolution of these materials, specifically after they were encapsulated with drugs and particles for controlled drug release makes their fabrication very challenging. Therefore, it is highly desirable to investigate novel materials with that can be used with a simple fabrication process for the generation of microneedles with sustained drug release profiles.

Microneedle patches for transdermal delivery are currently being explored in the forms of hollow, coated and dissolvable microneedles. Among them, the majority of microneedle patches tend to be coated un-dissolvable microneedles or dissolvable microneedles. During the earlier days of microneedle research, transdermal microneedles were coated with the desired drugs using a dip-coating approach and the drug formulations included the target drug, surfactant, and viscosity enhancer. These drug formulations were usually coated onto the microneedles consisting of robust materials such as metal or silicon. With these coated microneedles, the drugs were delivered across the skin barrier in small amounts, where the microneedles have limited drug loading capacity. In addition, the drug transfer mechanism from microneedles to the inner skin tissue is still not well understood, and there is a possibility that the delivered drug could be lost through the open space of microneedle insertion mark. Dissolving microneedles can overcome the limitations of drug loading capacity compared to the coated microneedles but has the same potential drug loss like in the case of coated microneedles due to incomplete insertion or dissolving of microneedles. Separable arrowhead microneedles could be a good alternative to prevent drug loss to fully embed the drug-loaded arrowhead in the skin. In addition, the microlancer integrated dissolving microneedles, which is a micropillar based system, was shown to achieve 97±2% delivery efficiency by fully embedding the microneedles with the aid of microlancer. However, sustained drug delivery has not been demonstrated by the current dissolving microneedles. There have been several attempts to achieve sustained drug delivery in the dissolving microneedle platform. A platform having encapsulated molecules within the microneedles which dissolved within the skin for bolus or sustained delivery was reported by Lee et al. See Lee et al., Dissolving microneedles for transdermal drug delivery, Biomaterials, 29(13), pp. 2113-24 (2008). Lysozymes were encapsulated with sulforhodamine B and molded with carboxymethyl cellulose mixture for creating dissolvable microneedles. This design provided drug release for several hours. Drug-loaded biodegradable poly(lactic-co-glycolic) acid (PLGA) microparticles in water-soluble poly(acrylic acid) (PAA) microneedle matrix was developed for long-term drug release. Another representative study by Chen et al. introduced embeddable chitosan microneedles onto supporting array for sustained delivery of encapsulated antigens to the skin. See Chen et al., Fully embeddable chitosan microneedles as a sustained release depot for intradermal vaccination, Biomaterials, 34(12), pp. 3077-86 (2012). The chitosan microneedles exhibited a sustained release of encapsulated antigens for several days in vitro via slow degradation. These representative dissolving microneedles for sustained drug delivery demonstrated controllable release profiles or long-lasting drug residue within the skin tissue, but additional fabrication steps including drug encapsulation or inclusion of particles are needed which makes the process unnecessarily complicated.

### SUMMARY

In one embodiment, a patch for therapeutic agent delivery across a biological barrier (e.g., skin) includes a base or substrate having a plurality of microneedles extending away from the surface of the base or substrate, wherein the base or substrate and the plurality of microneedles are formed from crosslinked gelatin methacryloyl (GelMA) and the plurality of microneedles optionally contain one or more therapeutic agents therein. The plurality of microneedles swell in response to absorbed fluid from the tissue that is breached by the plurality of microneedles. The patch or portions thereof is/are preferably biodegradable in some embodiments.

In another embodiment, a method of manufacturing a patch for therapeutic agent delivery across a biological barrier includes: providing a mold containing a plurality of needle-shaped cavities therein; applying a solution of un-crosslinked GelMA, one or more therapeutic agents, and a photoinitiator on or surrounding the mold. The mold is then subject to centrifugation to fill the needle-shaped cavities. Alternatively, ultrasound or vibratory motion is applied to the solution to aid in filling the mold cavities. Once the cavities have been filled, the mold is irradiated with light to crosslink the GelMA. The patch is then removed from the mold. The patch may be used directly or it may be secured to another backing material and then used.

In another embodiment, a method of using a patch formed from crosslinked GelMA is disclosed. The patch is placed on the tissue of a living mammal (e.g., skin tissue) such that the plurality of microneedles penetrate the epidermal layer of the skin tissue. One or more therapeutic agents contained in the plurality of microneedles are released over a period of time into the skin tissue. The release profile of the one or more therapeutic agents may be tuned by controlling the crosslinking degree of the GelMA. While the patch is described largely in the context of use with skin tissue the patch may also be used with other mammalian tissues. This may include, for example, cardiovascular tissue.

### BRIEF DESCRIPTION OF THE DRAWINGS

FIG. 1A illustrates a plan view of a patch for transdermal therapeutic agent delivery according to one embodiment. The patch includes a base or substrate and a plurality of microneedles that extend from a surface thereof.

FIG. 1B illustrates a cross-sectional view of a patch illustrating the base or substrate and the plurality of microneedles that extend from a surface thereof. A therapeutic material is illustrated being disposed within the microneedles and the base or substrate.

FIG. 1C illustrates a patch for transdermal therapeutic agent delivery being applied to skin tissue of a mammal (e.g., human).

FIG. 1D illustrates a plan view of an alternative embodiment of a patch for transdermal therapeutic agent delivery.

FIG. 2 schematically illustrates operations for the formation and use of GelMA microneedles for sustained drug delivery (in this embodiment DOX). The dissolvable microneedles are sharp enough to penetrate the skin barrier and are degradable, and microneedles can have the ability to release the loaded DOX into the transdermal space.

FIGS. 3A-3D illustrate the morphological characterization of the DOX-loaded microneedles. FIG. 3A illustrates optical images of the GelMA microneedles. FIG. 3B illustrates an optical image of DOX-loaded microneedles. FIG. 3C illustrates a SEM image of the GelMA microneedles. FIG. 3D illustrates a fluorescent microscope images of DOX-loaded GelMA microneedles (FITC, DOX, and merged). Scale bars are 500 μm.

FIG. 4A illustrates a graph of swelling ratio (%) as a function of crosslinking time. The error bars represent SD (n=3).

FIG. 4B illustrates representative swelling images of the DOX-loaded microneedles. The microneedles were scanned by confocal laser scanning microscopy (CLSM) (FITC modified GelMA, DOX, and merged) and scale bars are 250 μm.

FIG. 4C illustrates the effect of the duration of crosslinking on mechanical strength of the microneedles.

FIG. 5A illustrates a graph of percentage of degradation of the microneedles as a function of crosslinking time (15, 30 and 60 s). The error bars represent SD (n=3).

FIG. 5B illustrates histograms of drug (DOX) release rate (%) as a function of time for GelMA microneedles with different crosslinking times (0, 15, 30 and 60 s). The error bars represent SD (n=3).

FIGS. 6A-6E illustrate the penetration of the mouse cadaver skin by GelMA microneedles. Trypan blue was used to stain the penetrated skin (FIG. 6C). FIG. 6A is the untreated control. FIG. 6B is skin treated with microneedles only. FIG. 6C is skin treated with microneedles after Trypan Blue staining. FIG. 6D is an optical microscopy image of normal mouse skin before treating with microneedles. FIG. 6E is an optical microscopy image of mouse skin after treating with microneedles for 5 min.

FIG. 7A illustrates a graph of the viability percentage (%) as a function of crosslinking time of DOX-loaded microneedles for melanoma cell line A375. The in vitro cytotoxicity study measured cell viability by the MTT assay. Error bar represents SD (n=3).

FIGS. 7B-7F illustrate fluorescent microscopic images of A375 cells treated with different microneedles for 1 h (crosslinking time: 0, 15, 30, 60 s, respectively for FIGS. 7B-7E. FIG. 7F is the control cell without DOX treatment. The cells were stained by a Live/Dead assay.

FIG. 8A illustrates an optical image of GelMA microneedles.

FIG. 8B is a schematic illustration of GelMA microneedles (loaded with doxorubicin (DOX)) used as part of patch that is applied to mammalian skin. The microneedles pierce into the skin and the DOX contained therein is released into the dermal tissue in a sustained release.

### DETAILED DESCRIPTION OF THE ILLUSTRATED EMBODIMENTS

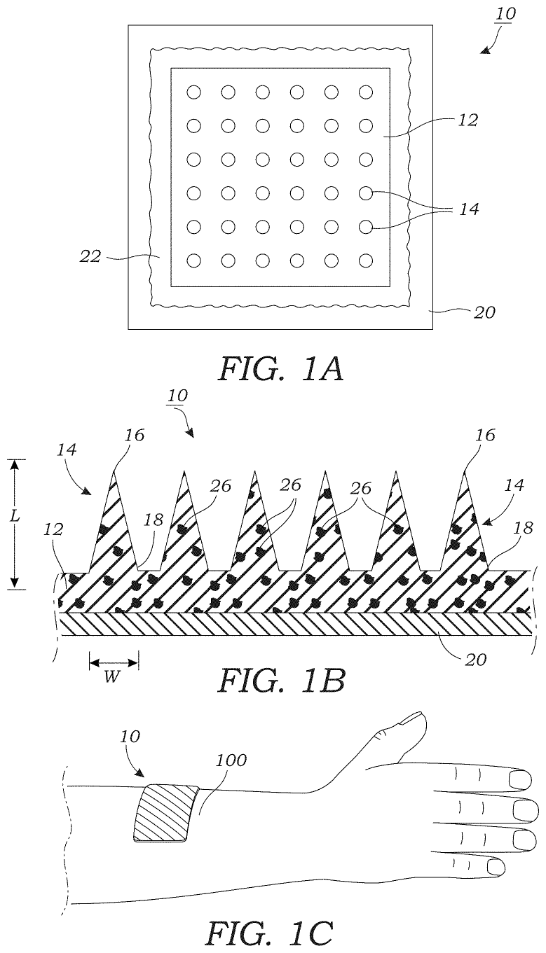

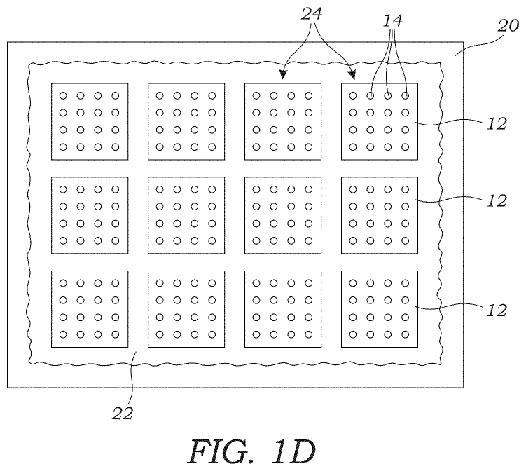

FIG. 1A illustrates plan view of a patch 10 for transdermal therapeutic agent delivery according to one embodiment. The patch 10 includes a base or substrate 12 that includes a plurality of microneedles 14 that extend or project from the substrate 12. The patch 10 may in some embodiments be partly or entirely biodegradable. The term biodegradable in the context of a biodegradable patch 10 refers to the base or substrate 12 and the microneedles 14 being formed from a material that is biodegradable. Other components such as the backing material 20 discussed below may not be biodegradable yet the patch 10 may still be referred to as being “biodegradable.” The plurality of microneedles 14 generally extend or project in a perpendicular direction from a surface of the base or substrate 12. The plurality of microneedles 14 may be arranged in a regular repeating array as illustrated in FIG. 1A or, alternatively, they may be arranged in a random pattern. In one embodiment, the plurality of microneedles 14 that are formed on the base or substrate 12 may have substantially similar shapes and sizes. However, in other embodiments, the plurality of microneedles 14 may have different shapes and/or sizes. For example, the perimeter region of the array or field of microneedles 14 that extend from the base or substrate 12 may be longer or have different shapes than those in the central region of the patch 10 to better secure the patch 10 to site of application.

In one particular embodiment, the microneedles 14, as their name implies, have a needle-like shape. For example, the microneedles 14 may include a sharpened tip 16 (seen in FIG. 1B) that aid in penetrating the epidermal layer of the skin tissue 100 (seen in FIG. 1C). The length (L) of the microneedles 14 may vary although typically the microneedles 14 extend less than about 1.5 mm from the base or substrate 12 (FIG. 1B). A typical length of the microneedles 14 is around 300-700 μm, although the dimensions may extend outside this range (e.g., around 10 μm to around 1,500 μm). The base 18 of the microneedle 14 is wider than the tip 16. Typically, the base 18 of the microneedle 14 may have a diameter or width (W) that is less than about 500 μm (e.g., 300 μm base and a height of around 700 μm) (FIG. 1B). The particular dimensions and shape(s) of the microneedles 14 are controlled by the particular construction of the mold that is used to form the patch 10, which is described more in detail below.

Still referring to FIGS. 1A and 1B, the base or substrate 12 which holds the microneedles 14 may be optionally bonded or otherwise adhered to a backing material 20 (e.g., through the use of an adhesive, chemical linking, or the like). The backing material 20 may be made from a woven fabric, a plastic material such as polyvinylchloride, polyethylene, or polyurethane, or latex. The backing material 20 may be flexible so that the patch 10, when applied, can conformally cover the tissue 100 (seen in FIG. 1C). Optionally, the backing material 20 may include an adhesive material 22 that covers all or a portion of the tissue-facing surface of the backing material 20. For example, adhesive may be formed on the backing material 20 around the periphery of the base or substrate 12 or the backing material 20 so that the base or substrate 12 may be secured in place to the surface of the tissue 100. The adhesive material 22 aids in securing the patch 10 to the tissue 100. The adhesive material 22 may include resins (e.g., vinyl resins), acrylates such as methacrylates epoxy diacrylates.

The base or substrate 12 and the microneedles 14 may be relatively rigid in the dry state. Because of this, in one alternative embodiment which is illustrated in FIG. 1D, multiple sub-patches 24 may be integrated into the backing material 20 to make the final patch 10. This may be useful for large coverage areas or curved surfaces that may pose a risk of breakage to the base or substrate 12. The various sub-patches 24, while generally rigid, are still able to conform to the surface of the tissue 100 (e.g., FIG. 1C) due the flexible backing material 20 which enables bending of the overall patch 10. Because individual sub-patches 24 are smaller in size these do not experience significant bending stresses which would otherwise cause a larger, rigid structure to break in response to bending and/or manipulation. Bending or flexing can occur within the backing material 20 between the locations of where the sub-patches 24 are located (e.g., between the rows and columns of sub-patches 24).

In one embodiment, with reference to FIG. 1B, the base or substrate 12 and the plurality of microneedles 14 are formed from crosslinked GelMA that contains one or more therapeutic agents 26 therein. There may be a single therapeutic agent 26 or a combination of different therapeutic agents 26 that work in concert together. The therapeutic agents 26 may include any number of drugs, medicaments, compounds, or pharmacological agents. For example, the therapeutic agents 26 may include a chemotherapeutic agent although it should be appreciated that a variety of different drugs or pharmacological agents may be loaded in the patch 10 (e.g., antibiotics, anti-inflammatory drugs, antiviral drugs, immunological agents (vaccines), therapeutic agents to treat pain, peptides, proteins, nucleic acids, cells, and the like). The therapeutic agent 26 may be dispersed throughout the entirety of the patch 10 including the base or substrate 12 and the plurality of microneedles 14 although in other embodiments the therapeutic agents 26 may be located only in the microneedles 14. The therapeutic agent 26 may be encapsulated within the crosslinked GelMA without any conjugation of chemical bond formed with the gel material. In other embodiments, the therapeutic agent 26 may be conjugated to the gel material via a chemical bond (e.g., covalent bond).

In addition, the microneedles 14 may contain a first therapeutic agent 26 while the base or substrate 12 may contain a second, different therapeutic agent 26. Alternatively, the microneedles 14 and the base or substrate 12 may contain the same therapeutic agent 26 but at different concentrations. Likewise, the base or substrate 12 may be formed with a different release rate than the release rate of the microneedles 14. This may be accomplished by forming the patch 10 using two different crosslinking operations where the microneedles 14 are crosslinked with a certain exposure time while the base or substrate 12 is crosslinked with a different exposure time (and thus degree of crosslinking). This can provide different release profiles of different or the same therapeutic agent(s) 26.

As explained herein, the base or substrate 12 and the microneedles 14 are preferably made from crosslinked GelMA. GelMA is a derivative of gelatin with modified methacrylamide or methacrylate groups. GelMA may be crosslinked by ultra-violet (UV) or visible light in the presence of a photoinitiator. It is a highly biocompatible material that is commonly used to support cell growth in tissue engineering. The existence of peptide moieties like arginine-glycine-aspartic acid (RGD) for cell attachment as well as for protease degradation makes GelMA a close mimic of the natural extracellular matrix (ECM). In addition, GelMA is a versatile material that can be easily functionalized with various bio-functionalities, such as by encapsulating different molecules including therapeutic agents, growth factors, and cytokines.

The microneedles 14 may have a number of different shapes and configurations including, for example, a pyramid, cone, cylindrical, tapered tip, canonical, square base, pentagonal-base canonical tip, side-open single lumen, double lumen, and side-open double lumen. The plurality of microneedles 14 swell upon breaching or penetrating the biological barrier and absorbing fluid from the surrounding tissue 100. The microneedles 14 may swell from about 100% to about 300% (wt. basis). The microneedles 14 swell and, in one embodiment, form a flexible hydrogel. The microneedles 14 provide a path for the therapeutic agent(s) 26 to pass through the biological barrier. In some embodiments, the microneedles 14 are also biodegradable and dissolve over time.

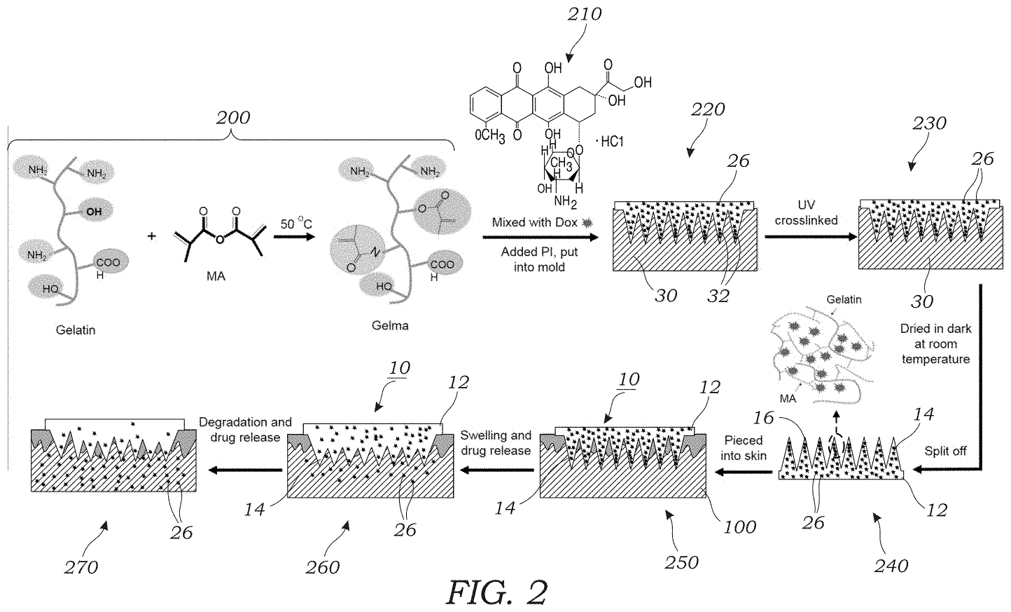

The patch 10 is manufactured or fabricated by providing a mold 30 such as that illustrated in FIG. 2 (e.g., micro-mold) containing a plurality of needle shaped cavities 32 therein. For example, the mold 30 may be formed from a polymer such as polydimethylsiloxane (PDMS). Commercially available microneedle molds 30 such as those made by Blueacre Technology Ltd. (Dundalk, Co Louth, Ireland) may be used. As seen in FIG. 2, in operation 200 the GelMa is formed using established protocols such as those disclosed in Yue, K., et al., Structural analysis of photocrosslinkable methacryloyl-modified protein derivatives. Biomaterials, 2017. 139: p. 163-171, and Yue et al., Synthesis, properties, and biomedical applications of gelatin methacryloyl (GelMA) hydrogels, Biomaterials, 2015; p. 254-271, which are incorporated herein by reference. Details regarding the formation of GelMa is described in detail herein.

The GelMa is mixed with the therapeutic agent(s) 26 and the photoinitiator (e.g., 2-hydroxy-4′-(2-hydroxyethoxy)-2-methylpropiophenone or Irgacure 2959) as seen in operation 210. Next, in operation 220, the solution of un-crosslinked GelMA that contains the one or more therapeutic agents 26 and a photoinitiator (PI) is then exposed to the mold 30. For example, the mold 30 may be placed in the solution and sonicated (e.g., subject to vibrational forces such as from ultrasonic waves) for a period of time to aid the solution to penetrate into the needle shaped cavities 32. Alternatively, or in addition to, the mold 30 with the GelMa/therapeutic agent 26 precursor solution is subject to centrifugation to aid in filling the mold cavities. For example, molds 30 may be placed in the wells of a well plate and a small (e.g., ˜100 μL of previously prepared GelMa precursor solution (with therapeutic agent 26) is loaded on top of the mold 30). The well plate may be centrifuged at 3,500 rpm for 15 minutes at around 37° C. to let the solution fully enter the mold 30.

Next, the mold 30 (which now contains the cast pre-cursor solution) is irradiated with light to crosslink the GelMA as seen in operation 230. The particular wavelength(s) used to crosslink GelMA may depend on the particular photoinitiator that is used. In some embodiments, visible light may be used to crosslink the GelMA. In other embodiments including those described in the experimental section herein used ultraviolet light (e.g., 350 mW/cm² UV light (360-480 nm)). The degree of crosslinking of the GelMA is controlled by the length of time that the mold is exposed to ultraviolet light (or other wavelength). Typically, the GelMa is exposed to ultraviolet light for between about 10 seconds and about 60 seconds. Additional crosslinking of the GelMA may be accomplished illuminating with ultraviolet light for longer than 60 seconds. For example, the mold may be irradiated with ultraviolet light for about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more seconds. It should be understood that crosslinking may take place in less or more time than the range set forth above. The mold 30 containing the now crosslinked GelMA is then subject to a drying operation (e.g., dried at around room temperature for about 24 hours). The base or substrate 12 having the microneedles 14 is then removed from the mold 30 as seen in operation 240. The base or substrate 12 having the microneedles 14 may be used directly as illustrated in operation 250 of FIG. 2 where the patch 10 is applied to the tissue 100 where the microneedles 14 pierce the tissue 100 (e.g., epidermal layer). The microneedles 14 and the base or substrate 12 swell in response to fluids contained in the tissue 100 and the therapeutic agent(s) 26 contained in the microneedles 14 and/or the base or substrate 12 are released in to the tissue 100 as seen in operation 260. After time, the patch 10 undergoes degradation which further aids in releasing the therapeutic agent(s) 26 into the tissue 100 as seen in operation 270. While FIG. 2 illustrates an embodiment in which no backing material 20 is used it should be appreciated that the backing material 20 may be bonded or adhered to the removed base or substrate 12 having the microneedles 14 (after operation 240).

In the embodiment described above the therapeutic agent(s) 26 are found in both the base or substrate 12 and the microneedles 14. In an alternative embodiment, the therapeutic agent(s) 26 may be only located in the microneedles 14. This may be accomplished in a two-step molding process where the precursor solution with the therapeutic agent(s) 26 is cast upon the mold 30 (e.g., a thin layer) to form the therapeutic agent(s)-containing microneedles 14. A second precursor solution that does not contain the therapeutic agent(s) 26 may be then be cast upon the microneedles 14 to make a base or substrate 12 that is free of therapeutic agent(s) 26.

### Experimental

A transdermal drug delivery patch 10 was developed using GelMA as the main base material. The microneedle 14 patches 10 were fabricated by micro-transfer molding and the anticancer drug doxorubicin (DOX) was loaded by one step molding, and crosslinked by UV irradiation as explained herein. The mechanical properties and drug release behaviors of the DOX-loaded microneedles 14 were evaluated. The efficacy of the DOX released from the GelMA microneedles 14 was demonstrated using a melanoma cell line A375.

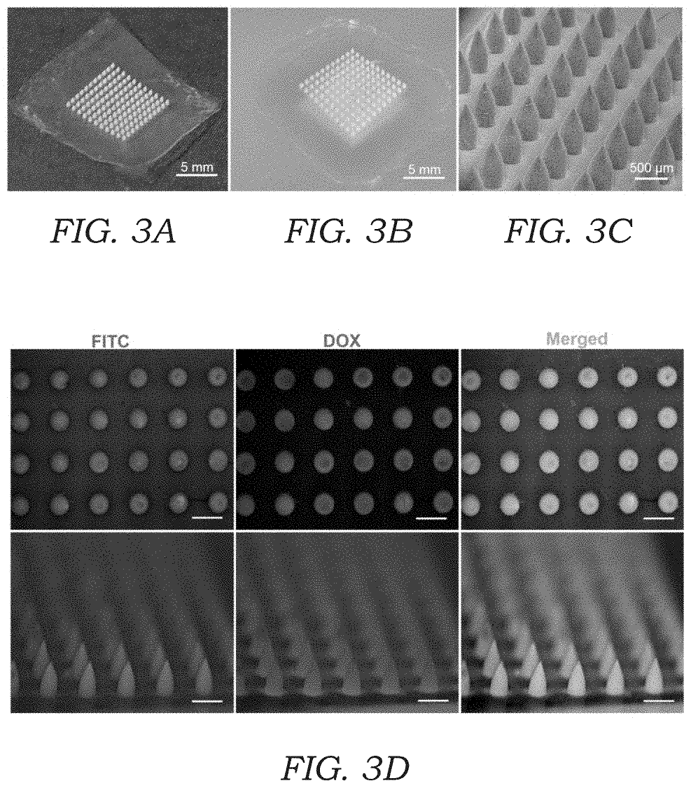

GelMA possesses superior biological properties, such as a high degree of biocompatibility, tunable biodegradability, and mechanical properties, making it a promising material for the fabrication of microneedles 14 containing one or more therapeutic agents 26. As shown in FIG. 2, the GelMA solution containing DOX was cast into a micro-mold 30 with an 11×11 array. After crosslinking by UV irradiation, the microneedle 14 containing patch 10 was solidified by drying. As shown in FIG. 2, an array of microneedles 14 with sharp tips 16 was fabricated from GelMA by the micro-mold casting method. While drug-free microneedles 14 exhibited a white color (FIG. 3A), after loading with DOX, the microneedles 14 turn to the color pink (FIG. 3B). SEM shows the detailed dimensions of the prepared microneedles 14 with a height around 600 μm and a base width of around 300 μm (FIG. 3C). By covalently modifying the GelMA with FITC, DOX encapsulation in the microneedles 14 was further characterized by fluorescent microscopy. As illustrated in FIG. 3D, the DOX therapeutic agent 26 was evenly distributed in the microneedles 14 and the base or substrate 12.

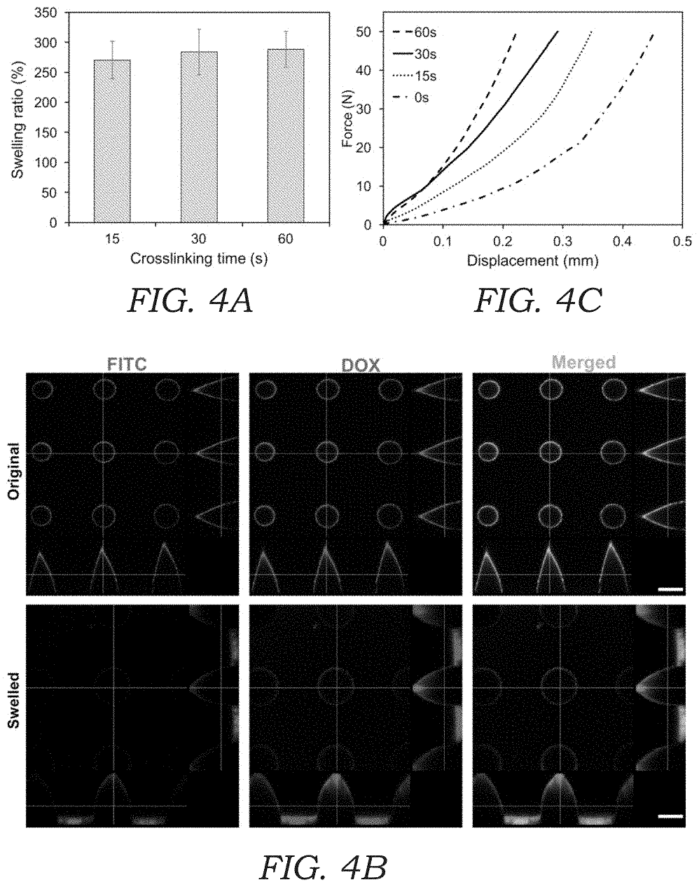

GelMA is hydrophilic porous material that is often applied in the form of a hydrogel. Applying the microneedles 14 onto the skin tissue 100 could lead to absorption of interstitial fluids into the microneedles 14. Swelling of the microneedles 14 could facilitate the release of the payload, and also has the potential to enhance the interaction of the microneedles 14 with the inserted cavity, stabilizing them into the punctured site. To investigate the effect of fluids on the swelling behavior of GelMA microneedles, DPBS was used to simulate the body fluid and the swelling ratio of GelMA microneedles 14 was measured. As shown in FIG. 4A, GelMA microneedles 14 with different crosslinking degrees (higher crosslinking time results in higher degree of crosslinking) all showed a swelling ratio of over 200%. Interestingly, extended crosslinking time resulted in a higher swelling ratio. This could be due to the relatively high solubility of GelMA microneedles 14 with low crosslinking degrees, where faster dissolution of GelMA caused higher weigh loss after 24 h incubation in DPBS. Next a confocal microscopy was used to observe the swelling of GelMA microneedles 14 (FIG. 4B). It was observed that the swelling ratio reduced the height of the microneedles 14 and increased their width. The unique formulation of GelMA-based microneedles 14 means that the structures have the ability to absorb interstitial fluids from the skin upon insertion and swell. The release rate of the therapeutic agent(s) 26 can be adjusted by controlling the degree of polymer crosslinking. A higher degree of crosslinking (increased crosslinking time) results in a slower release rate while a lower degree of crosslinking results in increased release rate. After the initial application as a tool to penetrate the stratum corneum barrier, the microneedles 14 become a rate controlling membrane.

Since the mechanical strength of the microneedles 14 is an important factor affecting the capability of the microneedles 14 to penetrate skin 100, the mechanical strength of the GelMA microneedles 14 was characterized. Microneedle arrays formulated using a super swelling formulation, with an 11×11 array were used to investigate the effects of compression tests on the heights of the individual microneedles 14 in the array. As shown in FIG. 4C, materials with higher crosslinking density (increased crosslinking time) required a higher amount of force to generate the same amount of compression, indicating that the increase of crosslinking time can significantly improve the mechanical strength of microneedles 14. Therefore, the amount of crosslinking in the GelMA microneedle 14 is an important parameter in determining the mechanical properties of the microneedles 14.

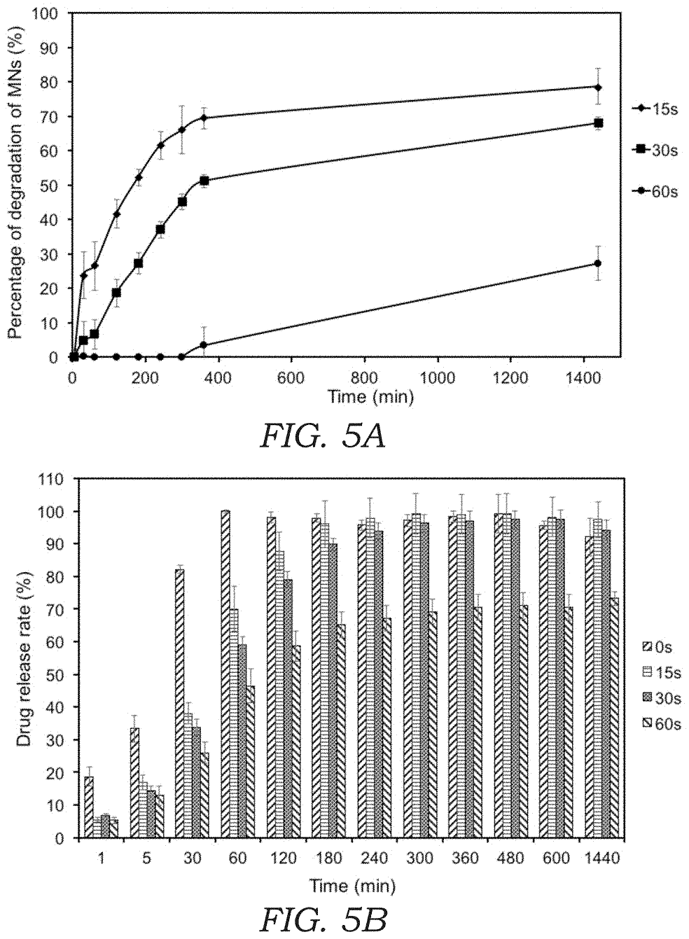

Besides swelling induced porosity of the GelMA microneedles 14, enzymatic degradation is another major factor in controlling the rate of release of the therapeutic agent(s) 26. To investigate protease-mediated degradation of GelMA and its associated effect on drug release, GelMA microneedles 14 were incubated in a collagenase solution. Changes in the wet weight of GelMA microneedles 14 were recorded to calculate the degradation rate of the microneedles 14. With reference to FIG. 5A, un-crosslinked GelMA microneedles 14 rapidly degraded within 60 min, while GelMA with a high crosslinking degree (60 s) showed only 20% of degradation after 1440 min.

Because DOX is a fluorescent molecule, the amount of released DOX from the GelMA microneedles 14 was tracked by testing DOX fluorescence in the supernatant of the solution incubated with DOX-microneedles 14 while being in the presence of the protease. The release kinetics of DOX from microneedles 14 were assessed over a period of 24 h. FIG. 5B shows that the higher crosslinking time resulted in decreased release rates. In contrast, microneedles 14 without crosslinking released over 80% of DOX in 30 min, while only a quarter of DOX was released in microneedles 14 that were crosslinked for 60 s. After being crosslinked for 60 s, microneedles 14 showed ˜50% release of the encapsulated DOX within the first 2 h, and then the remaining 20% was slowly released in the next 22 h, indicating that the DOX needed much more time to diffuse from crosslinked microneedles 14. The possible reason was that the porous structure of GelMA microneedles 14 became much compact after crosslinking, which trapped DOX inside the crosslinked network of microneedles 14. As time went by, microneedle patches 10 were gradually degraded by enzymolysis, and then the trapped DOX was subsequently released. Consequently, the lower release rate of DOX from crosslinked microneedles 14 could reduce the risk of cytotoxicity.

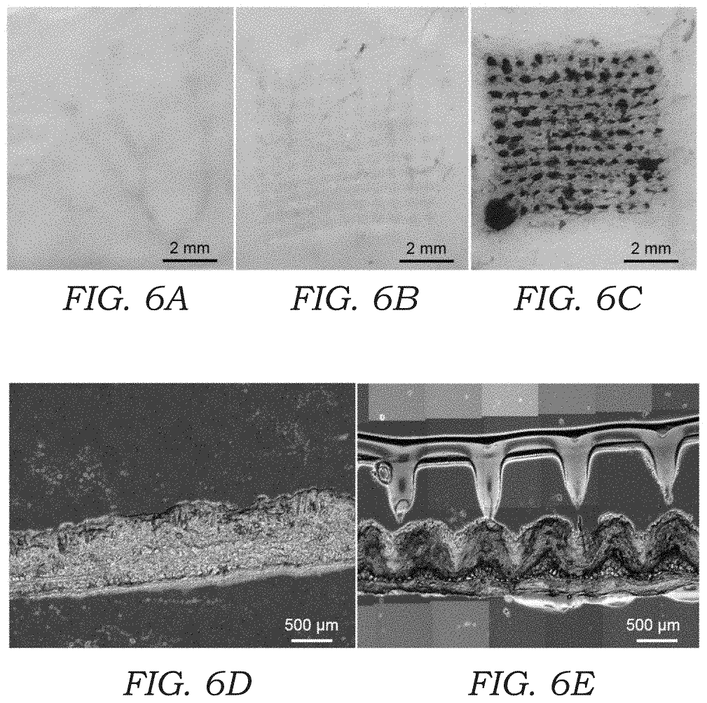

After investigating the mechanical properties of GelMA microneedles 14, the ability of the microneedles 14 to penetrate skin tissue 100 in a mouse cadaver skin model was tested. Mouse cadaver skin is widely used as a model for in vitro skin drug delivery studies, where the skin structure and permeability of the animal resemble that of humans. Compared with untreated skin (FIG. 6A), the microneedle-treated skin showed an 11×11 array of microchannels (FIG. 6B). To help visualize the micro-punctures, the treated skin was stained with trypan blue, which is a dye that preferably binds to damaged cells (FIG. 6C). This observation indicated a 100% penetration efficiency of the microneedles 14 into rat skin. As shown in FIG. 6D, the surface of the untreated mouse cadaver skin was smooth and is composed of epidermis, dermis, and hypodermis. Recurring microcavities with a depth of 400˜600 μm can be observed after the insertion of microneedles 14 as seen in FIG. 6E. After 5 min of insertion, the interstitial fluid of the mouse cadaver skin was absorbed by the microneedles 14 and resulted in the swelling of the microneedles 14. In spite of their swelling, the microneedles 14 retained their mechanical toughness in the hydrated state, which enabled their removal from the skin in an intact manner.

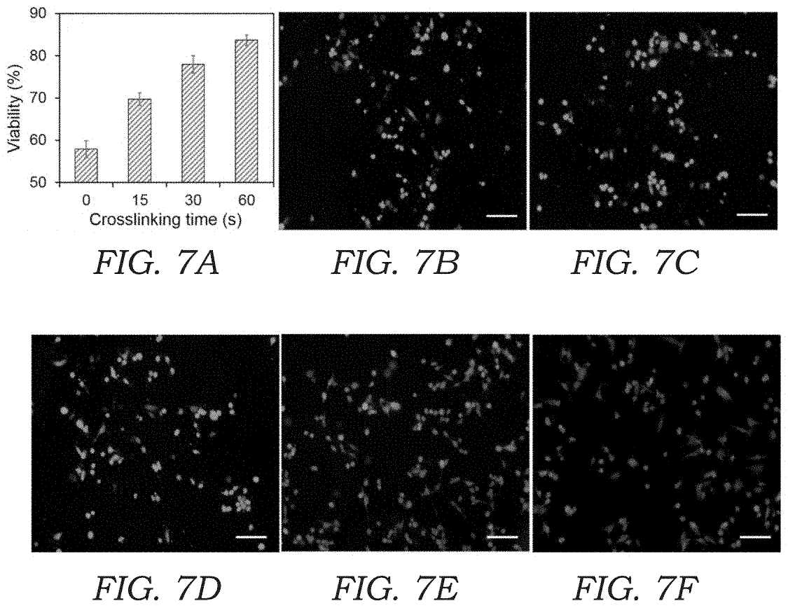

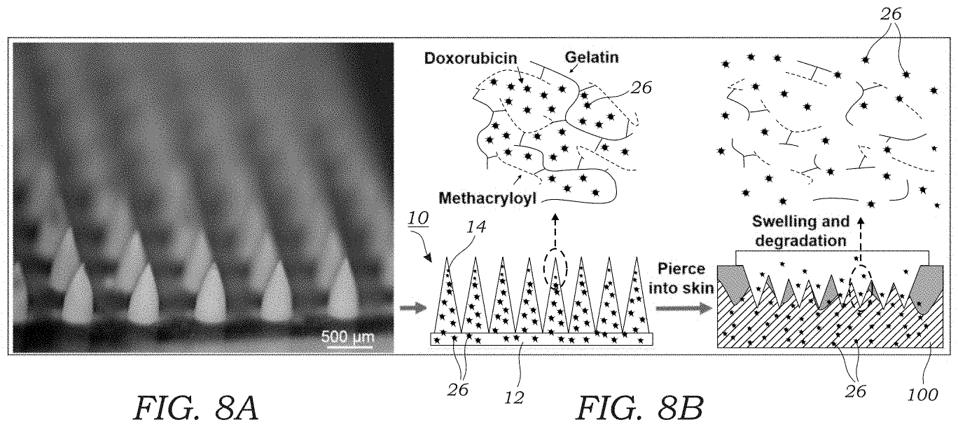

It was confirmed that the UV-mediated crosslinking process and the enzyme-mediated digestion of the GelMA scaffold did not influence the anticancer activity of the loaded DOX. A human melanoma cell line A375 was used as the model to investigate the anticancer efficiency of DOX released from the microneedles 14. After incubating microneedles 14 containing 10 μg of DOX with the plated cells for 1 h, the viability of A375 cells was examined using an MTT assay after 24 h. As shown in FIG. 7A, an inverse correlation between cell viability and crosslinking time was observed, which agreed with the fact that extended crosslinking times resulted in denser GelMA and reduced DOX release rates. The DOX-induced cell death was further investigated by a Live/Dead assay, where live cells metabolized calcein AM into the green fluorescent calcein, and dead cells with compromised membrane integrity were stained by the red fluorescent dye EthD-1. As shown in FIGS. 7B-7F, the cell death was observed in the DOX-treated cells. In addition, A375 cells treated by crosslinked microneedles 14 were observed less cell death than those treated by un-crosslinked microneedles 14, which was correlated with the viability assay (FIG. 7A). The possible reason was that microneedles 14 with more crosslinking time would release less DOX at the same releasing time (1 h).

Microneedle technology is promising for transdermal delivery of therapeutic agents 26 since it enables drugs to pass through the stratum corneum via microchannels in a minimally invasive manner. In general, the characteristics of the polymer and the casting medium can highly influence the properties of the microneedles 14. Here, GelMA was used as the base material to fabricate microneedles 14, and demonstrated their use for transdermal drug delivery by showing their skin penetration capability as well as the preservation of the therapeutic activity of the therapeutic agents 26 after release. The mechanical and material characteristics of GelMA microneedles 14 can be easily modulated by controlling their crosslinking degrees. Varying the crosslinking time (0 s to 60 s), the swelling ratio was found to change from about 250% to 290% (FIGS. 4A and 4B) and the mechanical strength was greatly enhanced (FIG. 4C). Compared with un-crosslinked GelMA microneedles 14, the degradation rate of GelMA microneedles 14 crosslinked for 60 s was sharply reduced, and the drug release profile was well controlled by varying the crosslinking time (FIGS. 5A and 5B). In addition, GelMA has been verified for its biocompatibility by being widely used in applications ranging from the food industry to medicine and pharmaceutical processing. This material has also shown superior cell viability for a wide range of cells. In vitro and in vivo studies with GelMA have shown that that GelMA hydrogels supported functional cell growths and promoted tissue healing with stable biocompatibility in animal models. In light of this, GelMA microneedles 14 has strengths in that it leaves no hazardous materials after biodegradation of GelMA.

GelMA microneedles 14 made of a crosslinked hydrogel containing DOX can penetrate across stratum corneum and reach the desired depths of the skin later. It is noteworthy to mention that GelMA microneedles 14 released their DOX cargo for a sustained period (up to 24 hrs.) and the delivery was carried out as the material slowly biodegraded at a slow pace. GelMA microneedles 14 may also be used to release therapeutic agents 26 over an even longer period of time (e.g., several days). GelMA microneedles 14 can be easily loaded with therapeutic agents 26 by using a mixing procedure. The release rate and/or release time of the therapeutic agent(s) 26 can be controlled by modulating the degree of crosslinking. Release times over a period of days, weeks, or even longer is possible by tuning the degree of crosslinking. Also, when the GelMA microneedle-containing patch 10 is applied to the tissue 100, there is no risk of loss of the therapeutic agent(s) 26 because the base or substrate 12 of the patch 10 covers top of the tissue 100 until the microneedles 14 deliver the therapeutic agent(s) 26.

GelMA is a promising material for the fabrication of a dissolvable microneedles 14 that can be used to deliver anticancer therapeutics (or other therapeutic agents 26). The GelMA based patch 10 that incorporates microneedles 14 exhibited sufficient mechanical strength to penetrate into mouse cadaver skin, and the microneedles 14 did not break or bend after the insertion. The GelMA microneedles 14 released their loaded therapeutics 26 through both swelling and enzymatic degradation of the scaffold. Compared with burst release that is often observed in some micro-needle formulations, the GelMA based microneedle patch 10 exhibited a gradual release of the loaded DOX, especially at higher crosslinking degrees (30 s and above). The controlled release was able to reduce the concern for burst release resultant toxicity. At high crosslinking degrees, a linear sustained release of DOX from the microneedles 14 was observed as oppose to a burst release. The DOX-loaded microneedles 14 have immense potential to function as a minimally invasive therapy for transdermal treatment of, for example, melanoma. For example, a patch 10 that contains an anti-cancer therapeutic agent 26 can be affixed over the region of skin tissue 100 that is cancerous where the anti-cancer therapeutic agent 26 is released over a period of time to the targeted site. As a versatile material in engineering tissue scaffolds, GelMA is also expected to be a promising platform for the delivery of both small molecule drugs and bio-macromolecular drugs including proteins, nucleic acids even cells.

GelMA preparation: GelMA was prepared as previously described in Yue et al. (2015), supra. Briefly, 10 g of type A porcine skin gelatin was added into 100 mL of DPBS preheated to 60° C. under constant stir. Methacrylic anhydride (8 mL) was gradually added and the reaction was kept under vigorous stirring for 3 h at 50° C. The reaction was stopped by adding a 5-fold volume of warm DPBS (40° C.). Residual salts and methacrylic anhydride were removed by dialysis in distilled water at 40° C. for 1 week using dialysis tubing with molecular weight cut-off of 12-14 kDa. After lyophilization for one week, GelMA in the form of white porous foam was obtained, which was stored at −20° C. for further use. FITC conjugated GelMA was obtained as follows: 1 g of GelMA was dissolved in 30 mL of DPBS and 0.1% FITC were mixed and the mixture was then reacted at 40° C. for 24 h in darkness, the conjugate was then dialyzed using dialysis tubing with a molecular weight cut-off of 12-14 kDa in distilled water at 40° C. The FITC modified GelMA in the form of yellow porous foam was obtained after lyophilization and was stored in darkness.

Preparation of DOX-loaded GelMA microneedles: For the microneedle 14 preparation, 0.4 g of GelMA was dissolved in 1.5 mL of DPBS solution at 50° C. Then 0.5 mL of DOX (400 μg mL⁻¹) and 10 mg of photoinitiator (Irgacure 2959) were added to the solution at 50° C. under vigorous stirring. The microneedle mold 30 was immersed into the prepolymer solution and sonicated for 1 h at 40° C., and then taken out of the solution and exposed to 350 mW (cm²)⁻¹ UV light (360-480 nm) for predefined exposure durations (0, 15, 30 and 60 s). Centrifugation may also be used to aid in filling the cavities of the microneedle mold 30 as described herein. The resulting microneedles 14 were manually removed from the mold after being dried in the dark for 24 h at room temperature.

Mechanical properties of microneedles: The mechanical strength of microneedles 14 was measured under dynamic force using a stress-strain gauge. The microneedle array was pressed against a stainless-steel plate on a low-force mechanical testing system (5943 MicroTester, Instron, USA), correlations between the applied force and deformation of the microneedles 14 were recorded. Initially, the microneedle tips were placed perpendicularly to stainless steel plate with a 1.5 mm distance and the maximum loading force was set at 50.0 N. Under a constant moving speed of stainless-steel plate (0.5 mm min⁻¹), the mechanical properties of microneedles 14 with different crosslinking times (0, 15, 30 and 60 s) were profiled. All tests were performed in triplicate.

Swelling, enzymatic degradation and drug release profile of DOX-microneedles: To analyze the swelling of the DOX-microneedles 14, UV crosslinked microneedle-containing patches 10 were incubated in DPBS for 24 h at 37° C. Incubated microneedles 14 were blotted to remove residual liquids, wet weight (W_{w}) of the microneedles 14 were recorded after microneedles 14 reached the equilibrium of swelling. The dry weights (W_{d}) were measured after freeze-drying. The swelling ratio was calculated as [(W_{w}−W_{d})/W_{d}]×100%. Three samples were used for the measurements to calculate the mean and standard deviations. In vitro degradation of microneedles 14 was also analyzed. Microneedles 14 were immersed in DPBS (5 mL) containing collagenase type II (2 U mL⁻¹) and incubated at 37° C. At the pre-determined time points, microneedles 14 were retrieved from the solution and the wet weights were recorded after blotting. The degradation ratio of microneedles 14 was calculated as (Wₜ/W₀)×100% (where Wₜ is residual wet weight at different time points and W₀ is the initial wet weight). All experiments were performed in triplicate. To investigate the drug release profiles of the microneedles 14, dried microneedles 14 loaded with DOX were immersed into 5 mL DPBS containing collagenase type II (2 U mL⁻¹). The samples were kept at 37° C., 100 μL of the DPBS was sampled at predefined time points, and the fluorescence of DOX (excitation 480 nm, emission 560 nm) was read using a Plate Reader (BioTek, USA). After the measurements, each sample was returned to the solution for drug release analysis. DOX was quantified using a calibration curve of DOX solutions with known concentrations (0.0055 μg mL⁻¹).

Skin penetration by the microneedles: To examine whether the microneedles 14 are mechanically strong enough to penetrate the skin, a mouse cadaver skin model was used. A patch 10 with microneedles 14 was pushed into the mouse cadaver skin by a compression force station (Instron, USA) with a force of 20 N for 5 s. Trypan blue, a dye that could stain damaged cell membranes, was then used to stain the penetrated tissue for 5 min. After removing excess trypan blue, the skin was imaged using an optical microscope (Zeiss, Sweden) to check for the sign of penetrating stratum corneum (blue dots). The cadaver skin of a mouse with microneedles 14 inserted was freshly frozen in OCT compound, and 10 μm thick cross-sectional slices were visualized on the Zeiss Axio Observer Z1 microscope (Carl Zeiss, Germany).

In vitro anticancer efficacy of the released DOX: In vitro cytotoxicity of the released DOX was evaluated using the melanoma cell line A375 as the model. Specifically, A375 cells were plated in 24-well plates (1×10⁶ per well) and incubated for 24 h. Microneedles 14 with different crosslinking degrees were added and incubated for 1 h. After that, the microneedles 14 were removed from the wells, and A375 cells were incubated for another 24 h. The effects of DOX on the metabolic activity of A375 cells in vitro were tested with a rapid colorimetric MTT assay. The absorbance of the wells was read at 570 nm with 630 nm as the reference. Live/dead staining was performed to visualize the viability of A375 cells after treatment with DOX released from the microneedles 14. The stained cells were then imaged by a fluorescent microscope (Zeiss, Sweden).

Statistical analysis: All data were shown as the mean±standard deviation (SD). Two-tailed Student's t-test was executed to evaluate the significance of the experimental data. Statistics was considered significant when p<0.05 or less.

While embodiments of the present invention have been shown and described, various modifications may be made without departing from the scope of the present invention. The invention, therefore, should not be limited except to the following claims and their equivalents.

1. A patch for therapeutic agent delivery across a biological barrier of living tissue comprising:
a base or substrate having a plurality of microneedles extending away from the surface of the base, wherein the base and the plurality of microneedles are formed from crosslinked gelatin methacryloyl (GelMA) and the plurality of microneedles contain one or more therapeutic agents therein.

2. The patch of claim 1, wherein upon application of the patch on biological tissue, at least the plurality of microneedles become wetted and swell, the plurality of microneedles providing a pathway for the one or more therapeutic agents to pass through the biological barrier and into the biological tissue.

3. (canceled)

4. The patch of claim 1, wherein the plurality of microneedles comprise sharpened tips.

5. The patch of claim 1, wherein the plurality of microneedles are biodegradable after insertion into the biological barrier.

6. The patch of claim 5, wherein a rate of degradation of the plurality of microneedles is controlled by the degree of crosslinking of the plurality of microneedles.

7. The patch of claim 6, wherein the degree of crosslinking is controlled by the time exposure to crosslinking light.

8. (canceled)

9. The patch of claim 1, wherein the biological barrier comprises skin.

10. The patch of claim 9, wherein the plurality of microneedles are disposed in the dermis or epidermis.

11. (canceled)

12. The patch of claim 1, wherein the plurality of microneedles exhibit a swelling ratio of at least 100% after the patch has been applied to skin tissue.

13. The patch of claim 1, wherein the plurality of microneedles exhibit a swelling ratio of at least 200% after the patch has been applied to skin tissue.

14. The patch of claim 1, wherein the one or more therapeutic agents are released into the tissue over a period of several days.

15. The patch of claim 1, wherein the one or more therapeutic agents are released into the tissue over a period of a week or more.

16. (canceled)

17. The patch of claim 1, wherein the plurality of microneedles have a length within the range of about 10 μm to about 1,500 μm.

18. (canceled)

19. The patch of claim 1, wherein the microneedles have diameter or width at the point of contact with the base that is less than about 500 μm.

20. The patch of claim 1, wherein multiple different therapeutic agents are contained in the plurality of microneedles.

21. The patch of claim 1, wherein the therapeutic agent comprises a chemotherapeutic agent, peptide, protein, nucleic acid, or cell.

22. The patch of claim 1, further comprising a backing material having an adhesive disposed thereon.

23. The patch of claim 1, wherein the plurality of microneedles absorb liquid from the tissue of the biological barrier and swell from about 100% (wt.) to about 300% (wt.).

24. The patch of claim 1, wherein the plurality of microneedles completely degrade in the tissue of the biological barrier.

25. The patch of claim 1, wherein the plurality of microneedles have a shape or configuration of: a pyramid, cone, cylindrical, tapered tip, canonical, square base, pentagonal-base canonical tip, side-open single lumen, double lumen, and side-open double lumen.

26. The patch of claim 1, wherein the concentration of GelMA is from about 5% (wt.) to about 40% (wt.).

27. A method of using the patch of claim 1 comprising placing the patch on live skin tissue of mammal such that the plurality of microneedles penetrates the epidermal layer of the skin tissue.

28-34. (canceled)
